(19) United States
(12) Patent Application Publication
(10) Pub. No.: US 2024/0034984 A1
(43) Pub. Date: Feb. 1, 2024

(54) CULTURE MEDIUM, METHOD FOR PRODUCING SAME, AND BACTERIAL CULTURE METHOD

(71) Applicants: Université de Bourgogne, DIJON CEDEX (FR); Institut National Supérieur des Sciences Agronomiques, de l'Alimentation et de l'Environnement, DIJON CEDEX (FR)

(72) Inventors: Laurent BENEY, DIJON (FR); Audrey RAISE, DIJON (FR); Sébastien DUPONT, BRETIGNY (FR); Eugénie HUILLET, Sevres (FR)

(21) Appl. No.: 18/258,201

(22) PCT Filed: Dec. 17, 2021
(86) PCT No.: PCT/FR2021/052397

§ 371 Date: Jun. 16, 2023

(30) Foreign Application Priority Data
Dec. 18, 2020 (FR) 2013684

(51) Int. Cl. C12N 1/20

(52) U.S. Cl. CPC C12N 1/20

(57) ABSTRACT

The present invention relates to a bacterial culture medium, to a method for producing the culture medium, and also to a method for culturing a bacterium, in particular an oxygen-sensitive bacterium. The culture medium of the present invention comprises, per liter of culture medium, the following components: from 20 to 40 g of potato peptones, from 2 to 5 g of anhydrous alkali metal acetate, from 3 to 7 g of a yeast extract, from 2 to 16 g of maltose monohydrate, from 0 to 1 g of a soluble L-cysteine salt, from 0.006 to 0.015 g of biotin, from 0.0005 to 0.0015 g of pantothenic acid or a salt thereof; and from 0.0015 to 0.0025 g of pyridoxal hydrochloride. The culture medium of the invention makes it possible to culture bacteria such as an intestinal anaerobic bacterium, F. prausnitzii, or an oxygen-sensitive bacterium or a strict anaerobic bacterium.

### TECHNICAL FIELD OF THE INVENTION

The present invention relates to a bacterial culture medium, to a method for manufacturing the culture medium, as well as to a method for culturing a bacterium, particularly effective for cultures of bacteria that are oxygen-sensitive, even very much so, or strict anaerobes. The culture medium of the invention makes it possible for example to culture intestinal anaerobic bacteria, for example Faecalibacterium prausnitzii (F. prausnitzii). The culture medium of the present invention makes it possible in particular to manufacture the new generations of probiotics, in particular those intended to prevent and treat chronic inflammatory diseases of the intestine in strict accordance with legislation on human or animal food.

In the text below, the term “Reference” followed by a digit refers to the list of the bibliographic references presented after the “Examples” section below.

### PRIOR ART

Originally, probiotics (“PBX”) were microorganisms, bacteria or yeasts, which, ingested in adequate quantities and potentially being integrated into certain food products for humans or animals, are supposed to confer a health benefit. The concept of probiotics was then extended to treatments for treating or preventing various conditions. It has indeed been demonstrated that probiotics have numerous advantages for human health and that they play a key role in normal digestive processes and in the maintenance of human or animal health. Agricultural applications of probiotics, in particular animal, fish, and plant production have gradually increased. However, there are a certain number of uncertainties concerning the technological, microbiological and regulatory aspects.

As an example, mention may be made of F. prausnitzii, the most abundant bacterium in the intestinal microbiota. It represents from 3.5 to 5% of the commensal bacteria. This bacterium is one of the major producers of butyrate, a short chain fatty acid, which is involved in gastrointestinal operations, and has numerous beneficial effects such as the defense of the intestinal mucosa, a motor effect on colon motility, a trophic effect on intestinal and colic epithelium, an effect on the defenses of intestinal mucosa by regulation of the secretion of mucus in the colon, making it possible to maintain a constant pH in the vicinity of epithelial cells, an effect on colorectal neoplastic cells, etc. (reference 1). However, this bacterium has extreme oxygen sensitivity, which makes it tricky to grow and handle. The need to grow it in an anaerobic chamber makes it technically difficult to produce and requires great expertise (reference 1). Furthermore, legislation in terms of human food is very strict, which further limits the current culture capacity of this microorganism, and more generally microorganisms having such oxygen sensitivities. Indeed, in particular, the culture medium must be devoid of compounds of animal origin.

The culture medium which was the most used until recently is the LYHBHI medium described in the document Sokol, H. et al. (2008), F. prausnitzii is an anti-inflammatory commensal bacterium identified by gut microbiota analysis of Crohn disease patients, Proc. Natl. Acad. Sei. U. S. A. 105, 16731-16736 (reference 2). However, this culture medium comprises compounds of animal origin and therefore cannot be used to produce probiotics in accordance with human dietary requirements.

There is therefore a real need for a culture medium that makes it possible both to culture bacteria that are sensitive or even extremely sensitive to oxygen or strict anaerobes, and to produce probiotics in accordance with strict human or animal dietary requirements.

### DESCRIPTION OF THE INVENTION

The aim of the present invention is precisely to meet these needs by providing a bacterial culture medium comprising, per liter of culture medium, the following components:
from 20 to 40 g of potato peptones,
from 2 to 5 g of anhydrous alkali metal acetate,
from 3 to 7 g of a yeast extract,
from 2 to 16 g of maltose monohydrate, for example from 2 to 12 g of maltose monohydrate,
from 0 to 1 g of a soluble salt of L-cysteine,
from 0.006 to 0.015 g of biotin,
from 0.0005 to 0.0015 g of pantothenic acid or a salt thereof; and
from 0.0015 to 0.0025 g of pyridoxal or a salt thereof.

According to the invention, the alkali metal acetate can be chosen from a sodium or potassium acetate, preferably anhydrous, for example sodium acetate or anhydrous potassium.

According to the invention, the yeast extract may for example be autolytic yeast extract, for example a lyophilized extract, for example resulting from the autolysis of yeast cells of Saccharomyces cerevisiae, grown on molasses, for example beetroot, soy or sugarcane. It may for example be a yeast extract with CAS number 8013-01-2.

According to the invention, the maltose monohydrate is preferably D-(+)-maltose monohydrate with CAS number 6363-53-7. The maltose is the source of sugar in the culture medium. Alternatively or in a mixture, cellobiose or other disaccharides, or the monosaccharides that compose them, may also be used.

According to the invention, the soluble salt of L-cysteine is preferably L-cysteine monohydrated hydrochloride. This component is a reducing component, which makes it possible to capture the molecular oxygen and to maintain a low redox potential in the culture medium.

According to the invention, the pantothenic acid may for example be the salt D-calcium pantothenate, the active form of vitamin B5.

According to the invention, the pyridoxal or the salt thereof may for example be pyridoxal hydrochloride or pyridoxal phosphate. This component can be replaced by pyridoxamine and pyridoxine.

The inventors found that this culture medium of the invention, which they have defined during their numerous experiments and work, with the particular trio of biotin, pantothenic acid or a salt thereof and pyridoxal, is particularly effective for cultures of bacteria that are oxygen-sensitive, even very much so, or strict anaerobes, even though it does not comprise any compounds of animal origin. The culture medium of the present invention provides excellent bacterial culture and growth performance, identical or even superior to culture media of the prior art comprising compounds of animal origin.

Furthermore, the culture medium of the present invention has numerous other advantages recorded by the inventors during the experiments, in particular: it does not foam, and therefore does not require the addition of anti-foaming agents; it does not precipitate, contrary to certain media of the prior art; and the number of components necessary for a quality bacterial culture is reduced, with only eight components.

Furthermore, the experiments carried out show that the bacterial performance in terms of production of probiotic compounds is preserved, or even improved. For example, in the case of cultures carried out with F. prausnitzii on the culture medium of the present invention, the measurements show that the production of butyrate in the medium is similar or even greater than that produced in media comprising compounds of animal origin, for example in comparison with a brain-heart infusion culture medium.

The culture medium of the present invention therefore advantageously makes it possible to produce new generations of probiotics from anaerobic bacteria, including extremely oxygen-sensitive ones or strict anaerobes, including but not limited to F. prausnitzii, that are intended to prevent and treat chronic inflammatory bowel diseases, in strict accordance with legislation on human or animal food.

The culture medium of the present invention can advantageously be used for culturing anaerobic, microaerophilic or strict, Gram-negative or positive anaerobic bacteria, for example for laboratory study purposes, seeking growth promoting treatments or intended to combat these bacteria, for example in food and/or in the field of medicine. Examples of bacteria that can advantageously be cultivated on the medium of the present invention include intestinal anaerobic bacteria, for example F. prausnitzii, Blautia, for example Blautia hydrogenotrophica and Blautia obeum.

The present invention also relates to a method for producing a culture medium according to the invention comprising:
(a) mixing potato peptones, anhydrous alkali metal acetate, yeast extract, and maltose monohydrate in a volume V1 of aqueous medium, preferably distilled water, thus obtaining a first mixture;
(b) adjusting the pH of the first mixture to a value of 7 to 8,
(c) heating the first mixture at the adjusted pH obtained in step (b) to a temperature of 110° to 130° C. for a duration of 15 to 30 minutes, followed by cooling to a temperature between 0° and 60° C.;
(d) mixing the soluble salt of L-cysteine, biotin, pantothenic acid or a salt thereof, and pyridoxal hydrochloride in a volume V2 of aqueous medium, preferably distilled water, thus obtaining a second mixture;
(e) sterilizing the second mixture; and
(f) mixing the first mixture cooled in step (c) with the second mixture obtained in step (e);
the amounts of the components and the volumes V1 and V2 being determined so as to obtain the concentrations defined above for the bacterial culture medium of the present invention.

According to the invention, step (c), which may be an autoclaving stage, is advantageously carried out after mixing with maltose.

According to the invention, step (c) of heating the first mixture at the adjusted pH obtained in step (b) to a temperature of 110 to 130° C. for a period of 15 to 30 minutes, followed by cooling to a temperature which may be from 0° to 60° C., can be carried out by autoclaving, according to any technique and with any apparatus known to a person skilled in the art to sterilize a culture medium without chemically modifying its components.

According to the invention, the maltose monohydrate may be added in step (a), that is before step (c), or in step (d) or (f), that is after step (c), preferably in step (a).

According to the invention, step (e) can be carried out as step (c), that is to say by heating the second mixture obtained in step (d) to a temperature of 110° to 130° C. for a period of 15 to 30 minutes, followed by cooling at a temperature of 0° to 60° C., or by filtration at 0.2 μm.

According to the invention, the step (f) of mixing the first and second mixtures is preferably carried out in sterile medium, for example under a microbiological safety station. According to the invention, the method may also comprise a degassing step (g),

for example under N₂H₂CO₂, for example lasting 15 minutes to 6 hours, following the volume to be degassed, for example for 2 to 6 hours for an industrial bioreactor. According to the invention, the degassing may also be passive degassing, for example lasting one night to several days, preferably in an anaerobic enclosure.

The present invention also relates to a method for culturing a bacterium comprising a seeding of a culture medium according to the invention by means of the bacterium, and a culture of the bacterium in the culture medium at a temperature allowing its growth.

A person skilled in the art knows the optimal bacterial growth temperatures or knows how to determine them, in particular from simple growth curves as a function of temperature. When they are intestinal bacteria, the culture temperature is generally 37° C.

According to the invention, the bacterium may be one of the aforementioned bacteria, for example an intestinal anaerobic bacterium, F. prausnitzii or an oxygen-sensitive bacterium or a strict anaerobic bacterium.

According to the invention, as the culture is for anaerobic microorganisms or even strict anaerobes, the culture is naturally carried out under anaerobic or strict anaerobic conditions. Anaerobic culture methods are known to a person skilled in the art and are applicable herein. Strict anaerobic bacteria can only be cultured in the absence of oxygen. This therefore requires limiting contact with ambient air. The culture medium can be incubated once inoculated in an oxygen-free environment, for example in an anaerobic chamber, which is a chamber of a Freiter chamber type, using gas mixtures to create the anaerobic conditions, for example N2 and/or CO₂, or in an anaerobic jar or plastic pouch that use chemical catalysts to create the anaerobic conditions, that is an atmosphere of less than 1% O₂ or even 0% O₂.

Other advantages will become more apparent in light of the following examples, given by way of non-limiting illustration, with reference to the appended figures.

### BRIEF DESCRIPTION OF THE FIGURES

FIG. 1 shows a curve measuring the change in the optical density at 600 nm of a culture medium according to the invention, as a function of the duration in hours (h) of culturing an anaerobic bacterium of the intestinal flora.

FIG. 2 shows a curve measuring the change in the pH of the culture medium according to the invention, as a function of the duration in hours (h) of culturing an anaerobic bacterium of the intestinal flora.

FIG. 3 shows a curve measuring the change in the colony-forming units (CFUs) per mL of a culture medium according to the invention, as a function of the duration in hours (h) of culturing an anaerobic bacterium of the intestinal flora.

FIG. 4 shows a curve measuring the change in the butyrate concentration in a culture medium according to the invention, as a function of the duration in hours (h) of culturing an anaerobic bacterium of the intestinal flora.

FIG. 5 shows a curve measuring the change in the optical density (OD), measured at 600 nm, from Blautia MB9 cultures in a culture medium according to the invention, as a function of the duration in hours (h) of culture.

FIG. 6 shows the curve measuring the change in the optical density at 600 nm of a culture medium according to the invention, as a function of the duration in hours (h) of culturing F. prausnitzii.

### EXAMPLES

### Example 1: Preparation of Culture Media According to the Invention

Table 1 below lists the components used in these example embodiments of the invention.

TABLE 1

| | | | Example |
|---|---|---|---|
| Component | | Example product | commercial |
| number | Component | used | reference |
| 1 | Potato | Ultra-light potato | Potato peptone |
| | peptones | protein hydrolysate | E210, |
| | | | Organotechnie |
| | | | (registered |
| | | | trademark) SAS |
| 2 | Anhydrous | Anhydrous sodium | reference S2889 |
| | alkali metal | acetate in powder | (commercial |
| | acetate | form, CAS number | reference), Sigma- |
| | | 127-09-3 | Aldrich |
| 3 | Yeast extract | Extract in powder | reference Y1000 |
| | | form obtained from | (commercial |
| | | Saccharomyces | reference), Sigma- |
| | | cerevisiae CAS | Aldrich |
| | | number 8013-01-2 | |
| 4 | Maltose | D-(+)-maltose | reference M5885 |
| | monohydrate | monohydrate from | (commercial |
| | | potato, CAS number | reference), Sigma- |
| | | 6363-53-7 | Aldrich |
| 5 | Soluble | L-cysteine | reference C7880 |
| | salt of | hydrochloride | (commercial |
| | L-cysteine | monohydrate, | reference), Sigma- |
| | | CAS | Aldrich |
| | | number 7048-04-6 | |
| 6 | Biotin | D-biotin in the form of | reference B4501 |
| | | lyophilized powder, | (commercial |
| | | CAS number 58-85-5 | reference), Sigma- |
| | | | Aldrich |
| 7 | Pantothenic | D-pantothenic acid | reference P2250 |
| | acid or a salt | hemicalcium salt | (trade reference), |
| | thereof | | Sigma |
| 8 | Pyridoxal | Pyridoxal | reference P9130 |
| | | hydrochloride | (trade reference), |
| | | | Sigma |

Table 2 below lists the various compositions of the components listed in Table 1 used in these example embodiments of the invention.

TABLE 2

| Component | 1 | 2 | 3 | 4 | 5 | 6 | 7 | 8 |
|---|---|---|---|---|---|---|---|---|
| Medium 1 | 20 | 3 | 7 | 12 | 0 | 0.006 | 0.0005 | 0.0015 |
| Medium 2 | 20 | 5 | 5 | 8 | 0.5 | 0.006 | 0.0005 | 0.0015 |
| Medium 3 | 40 | 2 | 3 | 2 | 0.5 | 0.006 | 0.0005 | 0.0015 |
| Medium 4 | 35 | 2 | 3 | 2 | 1 | 0.015 | 0.0015 | 0.0025 |
| Medium 5 | 30 | 3 | 5 | 4 | 0 | 0.008 | 0.0008 | 0.0020 |

The method for producing the culture medium was as follows:

For example, in Hungate tubes: dissolving components 1 to 4 in a volume of distilled water which was chosen as a function of the desired final volume. Part of the volume of distilled water was set aside, in this example 1%. The volume of distilled water was 1 L, and the volume set aside was 100 mL. Components 1 to 4 were added, each in the amount making it possible to obtain the abovementioned concentrations when the culture medium is finalized. Several media were manufactured, with indicated variations of concentrations. A first mixture was therefore each composed of the components 1 to 4. The pH of this first mixture was then adjusted to 7.4 with 10 M of soda.

The first mixture was heated to a temperature of 120° C. for 20 minutes in an autoclave in order to sterilize it. It was then left to cool. During this time, the components 5 to 8 are dissolved in the rest of the distilled water, here 100 mL (each in sufficient quantity to obtain the abovementioned concentrations when the culture medium is finalized), in order to obtain the second mixture. This second mixture was then sterilized by filtration at 0.2 μm under a microbiological safety station.

The second mixture is then added to the first mixture and the assembly mixed to homogeneity under the microbiological safety station, then subjected to degassing by means of a mixture of H₂CO₂ in N₂ (BlO-301, Air Liquide) for 4 hours.

For example in a bioreactor: compounds 1 to 4 are dissolved in distilled water and then this “basal” medium is placed in the bioreactor. The bioreactor is then autoclaved directly with the medium and the probes. Then, the filtered solution of vitamins and cysteine (compounds 5 to 8) is added to the bioreactor via a cannula under sterile conditions thanks to the use of a flame. Then, the medium is degassed directly in the tank for 30 minutes.

Various media were thus manufactured, including media 1 to 5 defined above.

### Example 2: Culture of F. prausnitzii and Performance

Different media manufactured as in example 1 were seeded with 2% to 4% by preculture volume of Faecalibacterium prausnitzii. These volumes correspond to seeds making it possible to obtain initial concentrations of bacteria in cultures between 1E+06 and 1E+08 CFU/mL.

In these examples, the F. prausnitzii used is the DSM 17677 strain (Hauduroy et al. 1937, Duncan et al. 2002 emend. Fitzgerald et al. 2018), from the German Collection of Microorganisms and Cell Cultures GmbH (Germany).

Before being used, all the media and all the liquid solutions coming into contact with the bacterium were actively degassed by bubbling the gaseous mixture N₂H₂CO₂ indoors for 4 h. This gas mixture was also used whenever it was necessary to establish or maintain anaerobiosis in a hermetic container or an enclosed space.

Once the media were seeded, they were placed in strict anaerobic culture at 37° C. in a Memmert oven, under the following conditions: anaerobiosis preserved by the Hungate tubes, 37° C. established owing to the oven, in a temperature bioreactor regulated by a bio-controller, anaerobiosis preserved by the passage of N₂H₂CO₂.

Appended FIGS. 1 to 4 show different culture performance measurements on the culture media of the present invention.

The curves shown in FIG. 1 were obtained from cultures carried out in Hungate tube seeded at 2% or 4% by volume. At each time of interest, 1 mL of culture was taken and then placed in a 2 mL spectrophotometry vessel. The optical density of the sample was then measured at 600 nm with a Helios Epsilon spectrophotometer (trademark—Thermoscientific). Each point corresponds to a culture in a different Hungate tube, used only to obtain the measurement at the given time.

In this figure, it can be seen that the growth of F. prausnitzii is sustained and enters the exponential phase more rapidly when the cultures are seeded at 4%.

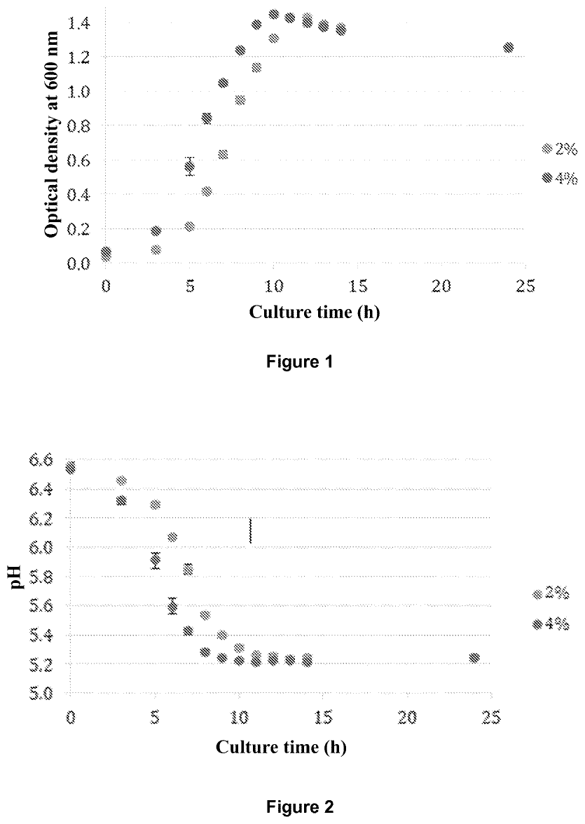

The curves shown in FIG. 2 were obtained from cultures carried out in Hungate tube seeded at 2% or 4% by volume. At each time of interest, the content of a culture in a Hungate tube was poured into a 40 mL sample collector and then the sample pH was measured using a CyberScan 500 pH-meter (trademark, Eutech Instruments—ThermoFisher Scientific, USA). The probe used was a HI 1053 probe (commercial reference—HANNA Instruments, France). Each point corresponds to a culture in a different Hungate tube, used only to obtain the measurement at the given time.

In this Figure, it can be seen that the pH decreases rapidly during the exponential culture phase and stabilizes around a value of 5.3 in stationary phase, for both series of culture. However, the drop in pH begins more quickly for the series of culture carried out with a seeding rate of 4%.

FIG. 6 shows the curve measuring the change in the optical density at 600 nm of a culture medium according to the invention, as a function of the duration in hours (h) of culturing F. prausnitzii. This curve was obtained under the same operating conditions as those used to obtain the curve of FIG. 1 with an inoculation ratio at 2%.

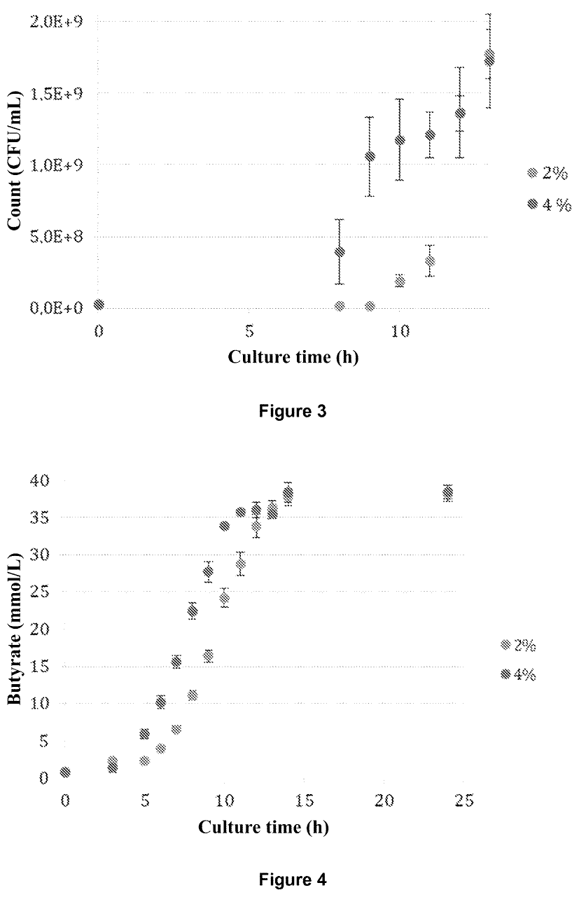

The curves shown in FIG. 3 were obtained from cultures carried out in a Hungate tube seeded at 2% or 4% by volume. At each moment of interest, the Hungate tubes were opened under a Bactron 600-2 anaerobic chamber (trademark—Sheldon Manufacturing, USA) and the culture bacteria concentration was evaluated by the colony forming unit (CFU/mL) method. Each point corresponds to a culture in a different Hungate tube, used only to obtain the measurement at the given time.

In this figure, it can be seen that the various media in accordance with the present invention that were tested made it possible to obtain about 2×10⁹ CFU/mL during cultures in Hungate tubes and in a useful 0.5 L bioreactor. However, the series of culture carried out with a seeding rate of 4% has a shorter latency phase and enters an exponential phase more quickly than the series of culture carried out with a seeding rate of 2%. Still, the maximum CFU/mL values reached remain identical.

The curves shown in FIG. 4 were obtained from cultures carried out in Hungate tube seeded at 2% or 4% by volume. At each time of interest, 1 mL of bacterial suspension was taken in a Hungate tube and then 100 μL of a 5% (v/v) solution of 85% orthophosphoric acid (commercial reference 20626.292, VWR) and 1% (m/v) of mercuric chloride (commercial reference 4187302, Merck) were added thereto. The samples were then homogenized and were stored at −20° C. The day of the analysis, the samples were thawed slowly and then 100 μL of an internal standard solution at 1% (m/v) of 4-methyl valeric acid (commercial reference 806088, Merck) were added to each sample. The samples were then mixed and then centrifuged for 10 min at 3800 g. The supernatant of each sample was then recovered using a syringe equipped with a needle and then filtered with a 0.2 μm regenerated cellulose filter (AF0-3203-12 commercial reference, Phenomenex (registered trademark). Each filtrate was then deposited in a Verex glass flask for gas chromatography (commercial reference AR0-3700-13, Phenomenex (registered trademark)) and sealed with a cap made of polytetrafluoroethylene (AR0-5710-13, Phenomenex (registered trademark)). The samples were analyzed by gas chromatography coupled to a flame ionization detector (GC-FID) using a Clarus 500 chromatograph (commercial reference, Perkin Elmer, France), equipped with a sampler and an automatic injector. The separation of the fatty acids was carried out on an Elite-FFAP capillary column (trademark) (0.25 mm×30 m×0.25 μm, N9316352, Perkin Elmer, France). For the analysis of each sample, 1 μL was collected and injected in “splitless” mode (without a partition ratio) into the column. The injection temperature was 165° C. and the carrier gas used was nitrogen, at a flow rate of 1 mL/min. The sample was then heated for 13 min at 135° C. in the furnace and then reached the flame ionization detector (FID), heated to 155° C. Between each injection, the syringe was rinsed with 1 μL of distilled water and 1 μL of sample to be analyzed. For each sample, the analysis was carried out in duplicate. The chromatograms were then analyzed using the software Azur (commercial reference—Perkin Elmer, France). The butyrate was identified by comparison of its retention time with that obtained during the passage of a solution of standards (carried out in the laboratory) analyzed under the same conditions. The retention time was around 6.31 seconds.

In this FIG. 4, it is noted that F. prausnitzii has indeed retained its capacity to produce butyrate, which demonstrates that it remains metabolically active and that it retains its probiotic effects in a culture medium in accordance with the present invention. The two series of culture make it possible to obtain the same maximum concentration of butyrate produced, reached during the start of the stationary phase.

### Example 3: Culture of Blautia MB9

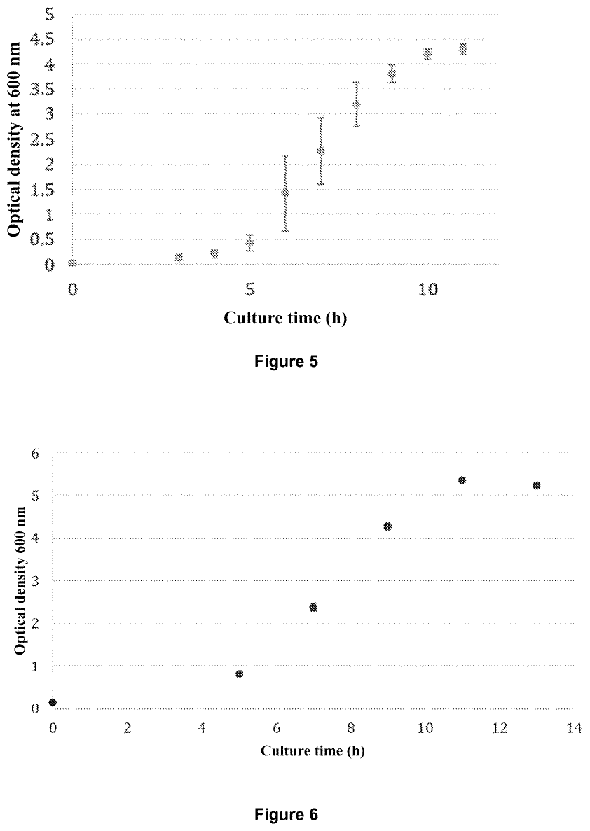

The curve shown in FIG. 5 shows the experiment carried out in this example. It was obtained from cultures of Blautia MB9 (Blautia obeum) in culture medium and under the conditions described in example 2. At each moment of interest, a volume of culture was taken and the optical density of the sample was then measured at 600 nm with the Helios Epsilon spectrophotometer (trademark—Thermoscientific). In this figure, it can be seen that the growth of Blautia is sustained. The exponential phase begins after 5 h of culture. The start of the stationary phase takes place after 10 h of culture. Similar results are obtained with Blautia hydrogenotrophica.

### BIBLIOGRAPHIC REFERENCES

Reference 1: Gastroentérologie clinique et biologique, Acides gras à chaîne courte: effets sur le fonctionnement gastro-intestinal et potentiel thérapeutique en Gastroentérologie, or “Short chain fatty acids (SCFA): effects on gastrointestinal function and therapeutic potential in Gastroenterology”, vol. 23, No. 6 and 7, Masson, Paris, July 1999, p. 761 and s.
Reference 2: Sokol, H. et al. (2008), Faecalibacterium prausnitzii is an anti-inflammatory commensal bacterium identified by gut microbiota analysis of Crohn disease patients, Proc. Natl. Acad. Sei. U. S. A.105, 16731-16736.

1. A bacterial culture medium comprising, per liter of culture medium, the following components:
from 20 to 40 g of potato peptones,
from 2 to 5 g of anhydrous alkali metal acetate,
from 3 to 7 g of a yeast extract,
from 2 to 16 g of maltose monohydrate,
from 0 to 1 g of a soluble salt of L-cysteine,
from 0.006 to 0.015 g of biotin,
from 0.0005 to 0.0015 g of pantothenic acid or a salt thereof; and
from 0.0015 to 0.0025 g of pyridoxal.

2. The culture medium according to claim 1, wherein the alkali metal acetate is anhydrous sodium acetate.

3. The culture medium according to claim 1, wherein the yeast extract is the yeast extract with CAS number 8013-01-2.

4. The culture medium according to claim 1, wherein the maltose monohydrate is D-(+)-maltose monohydrate with CAS number 6363-53-7.

5. The culture medium according to claim 1, wherein the soluble salt of L-cysteine is L-cysteine monohydrated hydrochloride.

6. The culture medium according to claim 1, wherein the pantothenic acid is the salt D-calcium pantothenate.

7. A method for producing a culture medium according to claim 1 comprising:
(a) mixing potato peptones, anhydrous alkali metal acetate, yeast extract, and maltose monohydrate in a volume V1 of aqueous medium, preferably distilled water, thus obtaining a first mixture;
(b) adjusting the pH of the first mixture to a value of 7 to 8,
(c) heating the first mixture at the adjusted pH in step (b) at a temperature of 110° to 130° C. for a duration of 15 to 30 minutes, followed by cooling to a temperature between 0° and 60° C.;
(d) mixing the soluble salt of L-cysteine, biotin, pantothenic acid or a salt thereof, and pyridoxal hydrochloride in a volume V2 of aqueous medium, preferably distilled water, thus obtaining a second mixture;
(e) sterilizing the second mixture; and
(f) mixing the first mixture cooled in step (c) with the second mixture obtained in step (e);
the amounts of the components and the volumes V1 and V2 being determined so as to obtain the concentrations defined in claim 1.

8. The method for culturing a bacterium comprising a seeding of a culture medium as defined in claim 1 by means of the bacterium;
and culturing the bacterium in the culture medium at a temperature allowing its growth.

9. The culture method according to claim 8, wherein the culturing is carried out under anaerobic or strict anaerobic conditions.

10. The culture method according to claim 9, wherein the bacterium is an intestinal anaerobic bacterium, Faecalibacterium prausnitzii or an oxygen-sensitive bacterium or a strict anaerobic bacterium.
